(19) United States
(12) Patent Application Publication
(10) Pub. No.: US 2011/0319971 A1
(43) Pub. Date: Dec. 29, 2011

(54) METHOD AND CONTROL SYSTEM FOR A TREATMENT BY SUBCUTANEOUS OR INTRACUTANEOUS IRRADIATION BY MEANS OF ELECTROMAGNETIC RADIATION

(75) Inventors: Jaouad Zemmouri, Genech (FR); Jean Ringot, Mons-en-Baroeul (FR); Benjamin Wassmer, Wattignies (FR)

(73) Assignee: OSYRIS MEDICAL, Villeneuve d'Ascq (FR)

(21) Appl. No.: 12/672,674

(22) PCT Filed: Aug. 7, 2008
(86) PCT No.: PCT/EP08/06532

§ 371 Date: Feb. 8, 2010

Related U.S. Application Data
(60) Provisional application No. 61/001,320, filed on Oct. 31, 2007.

(30) Foreign Application Priority Data
Aug. 16, 2007 (EP) 07370014.8

(51) Int. Cl. A61N 5/06

(52) U.S. Cl. 607/100

(57) ABSTRACT

To automatically control a treatment during which subcutaneous or intracutaneous irradiation by means of electromagnetic treatment radiation and possibly targeted electromagnetic radiation is carried out, the following steps are implemented: acquisition of several successive images l(t) of the treated zone by means of an external sensor (1), which is sensitive to the wavelength or the range of wavelengths of the electromagnetic treatment radiation or of the targeted electromagnetic radiation, the time interval (τ) between two successive images [l(t−1); l(t)] being known, detection and localisation in each image l(t) of a light spot p(t) corresponding to the irradiation spot (S) of the electromagnetic treatment radiation or the targeted electromagnetic radiation, calculation for each light spot p(t) of at least one of the following parameters: the energy [eᵢⱼ(t) or Eᵢⱼ(t)] supplied from the power P(t) of the electromagnetic treatment radiation and the time interval (τ) between two successive images [l(t−1); l(t)]; the displacement speed v(t) of the irradiation spot (S) from the positions of two light spots [p(t−1); p(t)] in two different images [l(t−1); l(t)] and the time interval between these two images [l(t−1); l(t)].

### FIELD OF THE INVENTION

The present invention relates to the field of treatment of the human or animal body by subcutaneous or intracutaneous irradiation by means of electromagnetic radiation. In this field, it relates in particular to a new technical solution for the control of the energy of the electromagnetic radiation applied in the course of a treatment, said solution finding its application in different types of subcutaneous or intracutaneous therapeutic or cosmetic endotreatments, such as lipolysis, endovenous therapy, skin remodelling or skin healing through heating the collagen present in the dermis and/or through thermal stimulation of the fibroblasts to accelerate the production of collagen in the dermis. The invention also has as its object a new method of skin remodelling or skin healing.

### PRIOR ART

In the field of therapeutic or cosmetic treatments of the human or animal body, to date different technical solutions are used based on subcutaneous irradiation of the zone to be treated by means of electromagnetic radiation, and based in particular on irradiation by means of electromagnetic radiation produced in the visible wavelength region for instance, using a continuous laser beam or a pulsed laser beam with different power levels. In these subcutaneous treatments, electromagnetic radiation is introduced under the skin to the zone to be treated, by means for instance of a hollow needle or a cannula, in which an optical fibre is inserted and linked to the adapted source of electromagnetic radiation, for instance a laser.

Treatments by subcutaneous electromagnetic radiation include primarily, but not exhaustively, lipolysis, which consists in destroying, in particular by the effect of heat, the adipose cells present in the hypodermis, by inserting into the hypodermis, at different depths, the distal extremity of the optical fibre, through which the electromagnetic radiation passes. They also include any endovenous therapy, in which electromagnetic radiation is produced in a vein. For laser lipolysis, the following publications can for instance be referred to: U.S. Pat. No. 6,206,873, U.S. Pat. No. 5,954,710 and US 2006/0224148. For endovenous laser therapy, publications U.S. Pat. No. 4,564,011, U.S. Pat. No. 5,531,739 and U.S. Pat. No. 6,398,777 can for instance be referred to.

A major difficulty of these treatments is linked to the risks of irreversibly destroying, through the effect of heat, non-targeted cells in the treated zone, or even in a zone adjoining the treated zone. This risk is dependent not only on the power and the wavelength of the electromagnetic radiation, but also and primarily on the speed with which the electromagnetic radiation spot is provided to the zone to be treated. The latter parameter of the speed of displacement, however, most often depends on a human manual action performed by the practitioner carrying out the treatment and is thus a significant source of risk.

Attempts to resolve this difficulty to date include efforts to control the energy of the electromagnetic radiation applied during treatment. In US patent application 2004/0199151, for instance, a solution is proposed based on measuring the speed of withdrawal of the optical fibre and on an automatic control of the laser power, as a function of the measured speed, so as to maintain a suitable constant treatment energy. Different solutions for measuring the displacement speed of the optical fibre are considered. For instance, specific marks made upon a certain length of the optical fibre are automatically detected or an optical speed measuring device, through which the optical fibre passes, is implemented. This solution has two disadvantages. On the one hand, the measuring means of the displacement speed of the optical fibre are positioned in the field of surgery, which brings about a problem of sterility of these measuring means. On the other hand, this solution does not enable the zone actually treated to be localised, and in particular does not enable the applied energy doses to be mapped at each point of the zone actually treated.

Other control solutions based on external detection of the skin temperature by means of an infrared sensor or by thermosensitive reagents applied on the skin have also been suggested. These solutions are not satisfactory, however, due in particular to the time required for the heat to propagate to the surface of the skin. Once the skin temperature threshold is reached and detected, it is generally too late and irreversible subcutaneous thermal lesions may already have been caused.

International patent application WO 2006/107522 suggests a solution for laser lipolysis, in which the laser beam is introduced into the hypodermis by means of a cannula/optical fibre unit. One objective in this publication is to protect the dermis against the destructive thermal effects of the laser beam by ensuring that the distal extremity of the optical fibre, upon firing, is not situated in the dermis, but rather in the hypodermis, at a sufficient distance from the dermis. To this effect, the depth of the laser shot is controlled by detecting, by means of an external optical sensor, the intensity of the light energy of the shot, which passes through the different layers (hypodermis, dermis, epidermis) and which is visible from the outside because of the sensor. The greater the intensity, the shallower the laser shot. This solution does not, however, enable the energy applied during treatment to be controlled and in particular does not enable the energy doses applied in each point of the zone actually treated to be mapped.

In addition to the above-mentioned subcutaneous treatments, there are also skin heat treatments of the non-invasive type, in the field of dermatology.

In particular, non-invasive heat treatments are implemented to heat the collagen present in the dermis of the skin and/or to thermally stimulate the cells (fibroblasts) producing collagen in order to stimulate collagen production by the fibroblasts in the dermis.

A significant application of these non-invasive heat treatments of the dermis is the remodelling of the skin through collagen in order to reduce or get rid of wrinkles due to ageing or to suppress unsightly aspects of the skin, so-called “orange peel skin”.

U.S. Pat. No. 6,659,999 and US patent application US 2003/0040739, for instance, suggest skin remodelling solutions through collagen based on external electromagnetic radiation of the skin by means of an exolaser, for instance.

The stimulation of collagen production by an external laser can also be used to obtain improved skin healing. A method of skin healing by means of a 815 nm laser is for instance described in the article “Laser Assisted Skin Closure (LASC) by using a 815-nm Diode-Laser System Accelerates and Improves Wound Healing” by A. Capon et al, Lasers in Surgery and Medicine 28:168-175 (2001).

During these heat treatments of the dermis, it is important that sufficient heating of the dermis is obtained, without however reaching a coagulation temperature (of the order of 60° C.), which would destroy the fibroblasts.

A disadvantage of these non-invasive treatments lies in the risks of burning the epidermis. In practice, to avoid these risks of burning, one is forced to combine this heat treatment with an external and local cooling of the epidermis.

### SUMMARY OF THE INVENTION

According to a first aspect, the invention aims to suggest a new technical solution for the automatic and real-time control of a treatment by subcutaneous or intracutaneous irradiation by means of electromagnetic radiation, with the aim of controlling the energy doses supplied to the treated zone and in particular of avoiding the supply of excessive energy doses or, conversely, the supply of energy doses that are too weak and ineffective for the treatment.

The invention thus has as a first object an automatic control method of a treatment, during which subcutaneous or intracutaneous irradiation by means of electromagnetic treatment radiation and possibly by means of targeted electromagnetic radiation is implemented. This control method comprises the following steps:
acquisition of several successive images l(t) of the treated zone by means of an external sensor (1), which is sensitive to the wavelength or in the range of wavelengths of the electromagnetic treatment radiation or the targeted electromagnetic radiation, the time interval (τ) between two successive images [l(t−1); l(t)] being known,
detection and localisation in each image l(t) of a light spot p(t) corresponding to the irradiation spot (S) of the electromagnetic treatment radiation or the targeted electromagnetic radiation,
calculation for each light spot p(t) of at least one of the following parameters: the energy [eᵢⱼ(t) or Eᵢⱼ(t)] supplied from the power P(t) of the electromagnetic treatment radiation and the time interval (τ) between two successive images [l(t−1); l(t)]; the displacement speed v(t) of the irradiation spot (S) from the positions of two light spots [p(t−1); p(t)] in two different images [l(t−1); l(t)] and the time interval between these two images [l(t−1); l(t)].

For the implementation of the invention, the irradiation spot detected by the sensor can be, depending on the circumstances, the irradiation spot of the electromagnetic treatment radiation or the irradiation spot of the targeted electromagnetic radiation. Nevertheless, the electromagnetic radiation detected by means of the sensor will preferably be the electromagnetic treatment radiation, since this is in practice more powerful than the targeted electromagnetic radiation.

A further object of the invention is a control system of a treatment by subcutaneous or intracutaneous irradiation by means of electromagnetic radiation as defined in claim 10, and a medical device as defined in claim 18 and including said control system.

According to a second aspect, the invention has as an object the proposition of a new skin remodelling or skin healing method through irradiation by means of electromagnetic radiation.

In a characteristic manner according to the invention, irradiation by means of electromagnetic radiation is carried out in the sub-dermal layer. This irradiation by means of electromagnetic radiation in the sub-dermal layer enables the collagen present in the dermis to be heated and/or the production of collagen to be stimulated by heating the fibroblasts.

To date, solutions for heat treatment enabling skin remodelling or skin healing to be obtained have most often been non-invasive. Invasive solutions have certainly also been suggested, in particular in U.S. Pat. No. 5,370,642, but in this case and up to the present, systematic attention has had to be paid to the optical fibre of the endocular laser (endolaser) penetrating into the hypodermis, to a sufficient depth in order for the energy to be supplied at a sufficient distance from the dermis and to avoid any risk of heat damage to the dermis and epidermis.

The invention has the distinction, on the contrary, of attempting to get closer to the dermis and of demonstrating that it is possible to carry out a heat treatment through irradiation by means of electromagnetic radiation supplied to the sub-dermal layer, which is effective for obtaining skin remodelling or skin healing by the collagen in the dermis, without causing irreversible heat damage to the dermis and epidermis.

### BRIEF DESCRIPTION OF DRAWINGS

Other characteristic features and advantages of the invention will appear more clearly upon reading the detailed description hereinafter of several embodiments of the invention given by way of non-limiting and non-exhaustive examples, said description being given with reference to the figures, in which:

FIG. 1 is a cross-sectional view of part of the human body showing the epidermis, the dermis and the hypodermis,

FIG. 2 shows, in a schematic manner, a control system according to the invention,

FIG. 3 is a block diagram of a medical device according to the invention,

FIG. 4 is a cross-sectional view showing an irradiation spot supplied to the sub-dermal layer, as well as the camera of a control system according to the invention, which is used to detect and localise this irradiation spot,

FIG. 5 is a mapping algorithm of the energy doses supplied for endovenous therapy,

FIGS. 5a, 5b and 5c represent the image displayed on a screen for the operator at different stages of the treatment method, during the implementation of the steps of the flowchart of FIG. 5,

FIG. 6 is a mapping algorithm of the energy doses supplied for a treatment of the lipolysis type,

FIG. 7 is an algorithm of the control of the displacement speed of the irradiation spot.

### DETAILED DESCRIPTION

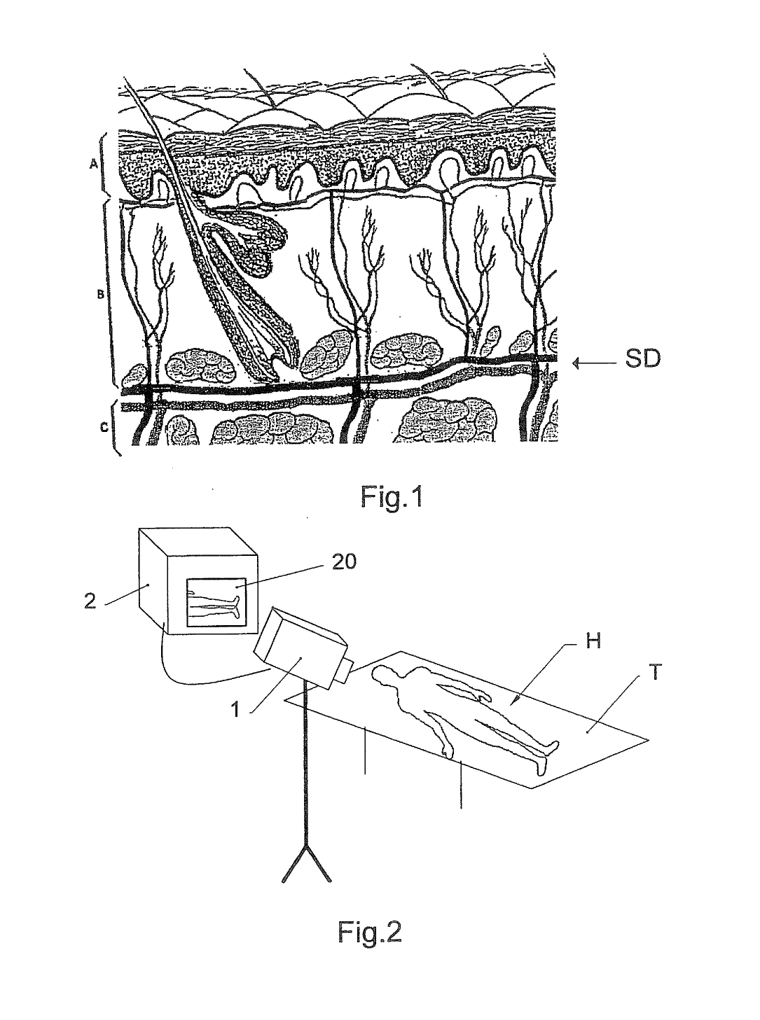

With reference to FIG. 1, the skin is formed of two main layers: a thin superficial layer A, composed of epithelial tissue and commonly called the “epidermis”; a deep and thicker layer B commonly called the “dermis”. Underneath the skin, the superficial fascia C, also called the “hypodermis”, attaches the dermis to the underlying organs and tissues. The SD layer situated immediately underneath the dermis, at the interface of the dermis B and the hypodermis C, is generally referred to as the sub-dermal layer.

To perform subcutaneous laser treatment, for instance to destroy the adipose cells present in the hypodermis (lipolysis), usually an optical fibre is inserted into the hypodermis C, by means of a cannula or hollow needle, after having made a small incision in the epidermis A and the dermis B, where appropriate. This optical fibre is linked at its proximal extremity (the opposite end to the distal extremity of the fibre inserted underneath the skin) to a pulsed or continuous laser source. This laser source can also be replaced by any other type of source enabling the supply of an appropriate electromagnetic treatment radiation, and for instance by a source composed of one of several high-power diodes. Once the optical fibre has been inserted, in order to carry out the treatment, the practitioner displaces the optical fibre while performing laser shots. Two principal techniques are implemented. A first technique consists in carrying out a withdrawal of the fibre in a discontinuous manner, and in performing a longer or shorter laser shot (continuous or pulsed) at each stop. A second technique, more commonly used because it is quicker, consists in carrying out a continuous withdrawal of the fibre at a substantially constant speed and, during this continuous withdrawal, in performing a laser shot (continuous or pulsed) without interruption. In practice, to cover the entire area to be treated, the practitioner must insert the optical fibre several times, in order to position initially the distal extremity of the optical fibre in different points of the zone to be treated and to carry out several withdrawal operations of the optical fibre with laser shots (without necessarily taking out the distal extremity of the optical fibre).

In a similar manner, to perform endovenous therapy by laser or equivalent, it is usual to insert an optical fibre into a vein by means of a cannula or hollow needle, and to treat the inside of the vein by performing laser shots according to one or the other of the above-mentioned discontinuous or continuous techniques.

The system and method of control according to the invention, of which one embodiment will be described in more detail shortly, enables an advantageous control, in this type of endotherapy through irradiation by means of electromagnetic radiation, of the energy of the electromagnetic radiation supplied to the treated zone.

It should be underlined that to date, in the case of subcutaneous laser treatments, in order to avoid the risk of burning of the dermis B and the epidermis C, systematic attention is paid to the insertion of the distal extremity of the optical fibre into the hypodermis to a depth in the hypodermis that is sufficient for the laser shot or equivalent not to be performed in immediate proximity to the dermis B. Because of the control of the energy of the electromagnetic treatment radiation obtained by means of the invention, it is now possible to perform new subcutaneous treatments in proximity to the dermis or in the so-called “sub-dermal” layer, at the interface between the dermis and the hypodermis, even to perform intracutaneous treatments, by positioning the distal extremity of the optical fibre in the dermis.

FIG. 2 shows, in a schematic manner, a patient H laid out on an operating table T. On this drawing, the means enabling endotherapy through irradiation by means of electromagnetic radiation (treatment laser, targeted laser, optical fibre and cannula for insertion and guiding of the optical fibre) are not represented.

The control system according to the invention includes a camera 1, enabling a plurality of images l(t) of the treated zone to be successively obtained, at a predefined capturing speed (time interval between two images l(t)).

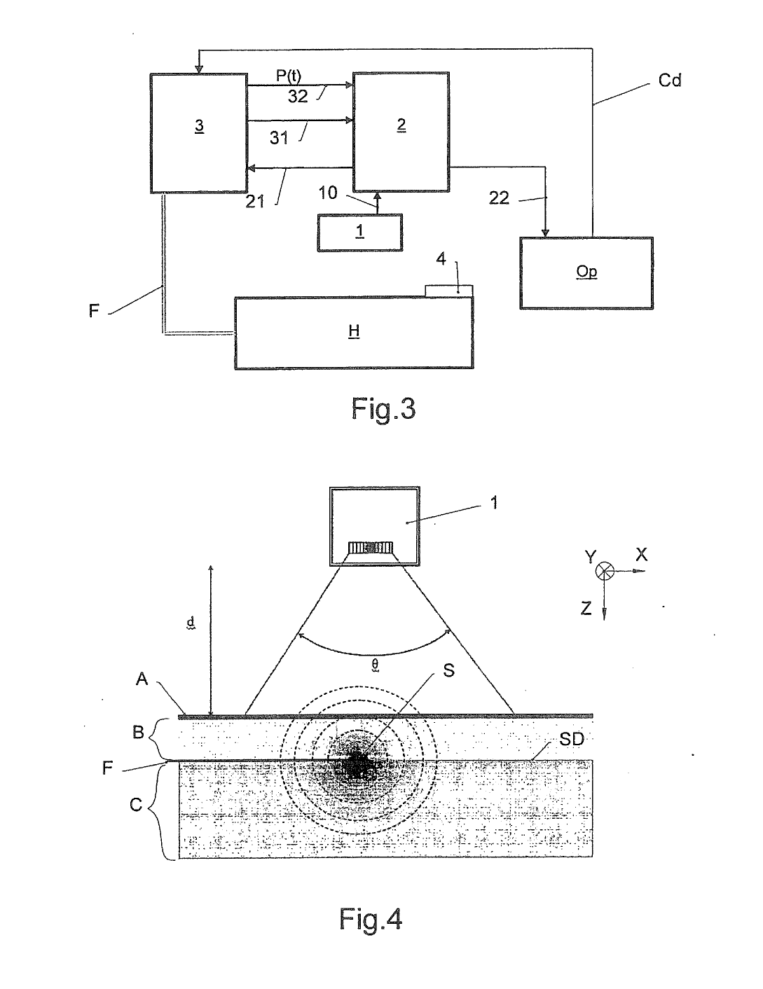

FIG. 3 shows a source 3, which enables electromagnetic treatment radiation to be supplied and which is linked to the optical fibre F, the distal extremity of which is inserted into the body of the patient H. This source 3 is a laser, for instance. The operator Op (practitioner) carrying out the treatment can, in the usual manner, control the triggering and manual stopping of a shot by means of a control Cd.

For each shot, the electromagnetic radiation produced can be of the pulsed or the continuous type.

If appropriate, the operator Op can also regulate the power P(t) of the source 3. In the present text, “power P(t) of the source 3” designates the average power of the source. In the case of continuous electromagnetic radiation, the power P(t) is equal to the instantaneous power of the electromagnetic radiation supplied by the source 3. In the case of electromagnetic radiation of the pulsed type, the average power P(t) is equal to the energy supplied during one second. For instance, for a laser of instantaneous power (Pₚₑₐₖ) of 1000 W and having a pulse width (T) of 150 μs with a repetition rate (f) of 40 Hz:
the energy E is equal to 6J (E=Pₚₑₐₖ×T×f) and
the average power P(t) is equal to 6W (P(t)=E/1 s).

Similarly, in a known manner, the medical treatment device can include a second electromagnetic radiation source (for instance a second laser source), which is also linked to the optical fibre F and which enables a second, so-called targeted, electromagnetic radiation to be supplied. This targeted electromagnetic radiation is generally of a weaker power than the electromagnetic treatment radiation supplied by the source 3. This targeted electromagnetic radiation is used to localise the subcutaneous or intracutaneous zone that is to be treated by the electromagnetic treatment radiation.

With reference to FIGS. 2 and 3, the camera 1 is linked to electronic treatment means 2, for the automatic treatment of the signal 10 supplied by the camera. These electronic treatment means 2 preferably include visualisation means 20 (screen 20/FIG. 1) enabling the image of the treated zone to be displayed to the operator Op (practitioner) and also, as will be shown more clearly later on, enabling a mapping of the energy doses supplied to the treated zone to be superimposed on this image.

With reference to FIG. 2, the electronic treatment means 2 also communicate with the source 3. More particularly, the electronic treatment means 2 are informed by the source 3, by means of signal 31, whether a shot is in progress or not (source 3 activated or not). The electronic treatment means 2 are also informed by the source 3, by means of signal 32, of the average power P(t) of the shot, said power can if necessary be regulated by the operator Op.

In the particular example of FIG. 3, the electronic treatment means 2 are also designed to control the stopping of the source 3 by means of a control signal 21 and to provide a warning signal 22 (audio and/or visual) to the operator Op, for instance if the energy supplied to a point reaches a predefined energy threshold.

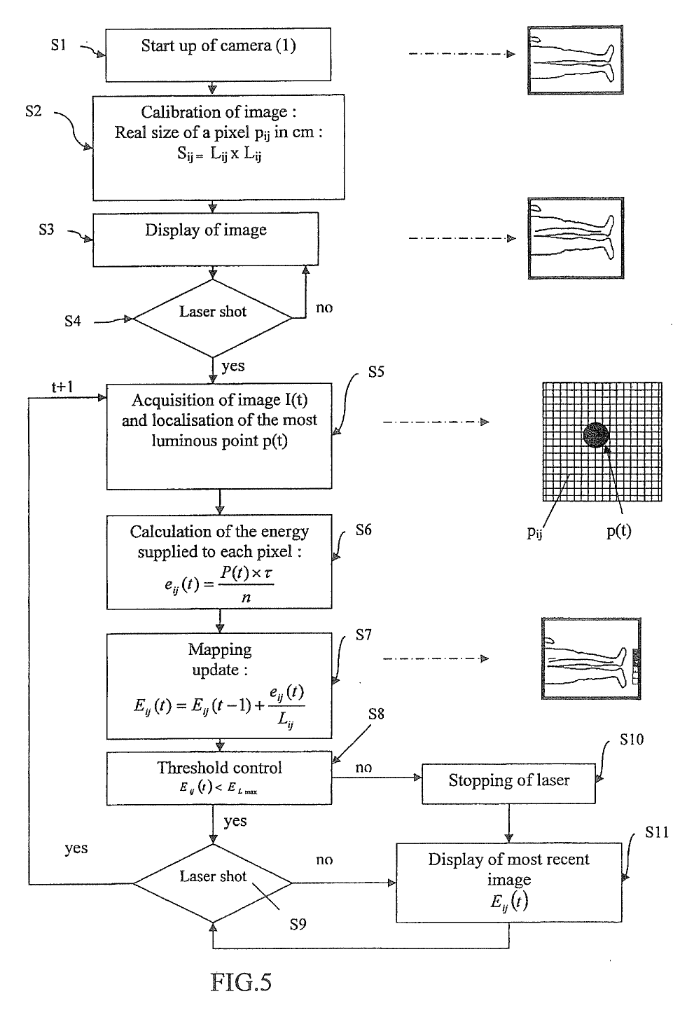

FIG. 4 shows, in a schematic manner, a cross-section showing an optical fibre F inserted into the sub-dermal layer (between the dermis B and the hypodermis C), during a shot.

The camera 1 is positioned at a distance (d) from the treated zone and shows a predefined viewing angle θ. Preferably, but not necessarily, during the course of treatment, the camera 1 is fixed, the distance (d) and the viewing angle θ being regulated so that the field of vision of the camera 1 covers the entire surface of the zone to be treated. Nevertheless, in another embodiment, the camera 1 can be mobile in order to cover the entire zone to be treated.

The camera 1 is furthermore chosen such that it is sensitive to the wavelength or the range of wavelengths of the electromagnetic radiation that is to be detected.

Preferably, the detected electromagnetic radiation is the electromagnetic treatment radiation supplied by the source 3. Nevertheless, for the implementation of the invention, the detected electromagnetic radiation can also be the targeted electromagnetic radiation.

In the rest of the description, the control of the treatment through irradiation is based on a detection by the camera 1 of the irradiation spot (S) of the electromagnetic treatment radiation supplied by the source 3. In a further embodiment, the control of the treatment by irradiation can be based on a detection by the camera 1 of the irradiation spot (S) of the targeted electromagnetic radiation.

In the majority of cases, the treatment is implemented by a laser or equivalent source having an emission wavelength in the visible range, generally between 600 nm and 1400 nm for the most commonly used lasers. Consequently, most of the time, the camera 1 will be chosen so that it can detect an electromagnetic radiation in the visible or near infrared region. The camera is for instance a matrix camera with charge transfer sensors, generally called a CCD camera (on the basis, for instance, of photosensitive silicon cells in the visible region or of germanium in the near infrared region).

The invention is not, however, limited to this 600 nm-1400 nm range of wavelengths, but can be implemented with radiations outside of this range, for instance with a HF (microwave) or RF (radiofrequency) radiation. In this case, the sensor will be adapted to the radiation wavelength (for instance, matrix sensor based on Schottky diodes).

By way of a non-limiting example, the control method according to the invention can be implemented with a CMOS 500×582 pixel camera enabling an image to be obtained every 0.04 seconds, a viewing angle of θ=70° and a distance (d) between the camera and the epidermis A of 20 cm. The surface visible via the camera in this case is 784 cm² (28 cm×28 cm). Each pixel thus corresponds to a surface of 0.27 mm² (0.52 mm×0.52 mm).

During a shot, at the distal extremity of the optical fibre F, the laser beam, when exiting from the optical fibre, forms an irradiation spot S (FIG. 4), the energy of which is at its highest in the centre (in immediate proximity to the exit of the optical fibre F) and the energy of which decreases as it moves away from the exit of the optical fibre F. The electromagnetic radiation of this irradiation spot S passes through the different layers of the skin (dermis B and epidermis A) and is detected by the camera 1. The camera 1 thus enables the position of the irradiation spot S to be detected through the skin and to be followed in time in a plane (X, Y) substantially parallel to the surface of the epidermis A (FIG. 4) and substantially perpendicular to the direction Z (direction corresponding to the depth).

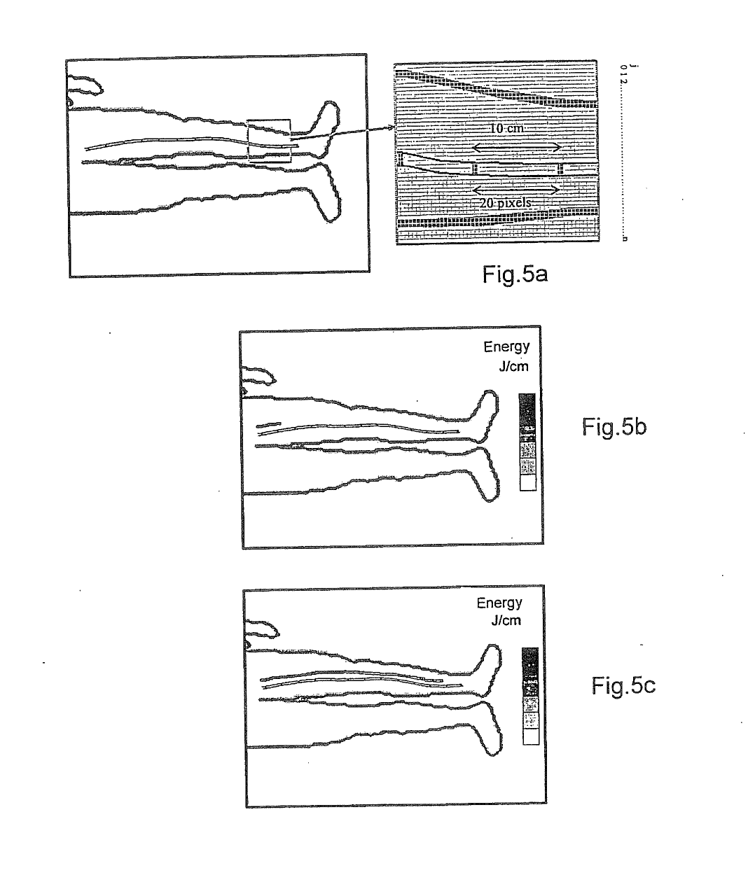

The electronic treatment means 2 include a treatment unit, which is programmed to carry out an algorithm, of which several embodiments are shown in the flowcharts of FIGS. 5 to 7, respectively. These flowcharts of FIGS. 5 to 7 are given by way of non-limiting and non-exhaustive examples of the invention.

### Example of a Mapping Algorithm for Endovenous Therapy—FIG. 5

This algorithm enables the mapping of the linear energy doses supplied in the course of endovenous therapy, during which the optical fibre F is inserted inside a vein and is withdrawn from the vein, either with a continuous movement, during which a shot is carried out without interruption by the operator (Op), or with a discontinuous movement, during which a plurality of successive shots are carried out by the operator (Op) at different positions of the distal extremity of the optical fibre F.

Steps S1 to S3 are calibrating steps of the camera 1, prior to the realisation of the endovenous therapy.

### Step S1:

During this first step, the operator Op starts up the camera 1.

### Step S2:

Then, the operator Op positions a calibration scale 4 (for instance, a graduated ruler with graduations spaced by a known distance d1) on the zone to be visualised. The electronic treatment means 2 obtain a real image of the zone to be treated (FIG. 5a/legs of the patient H with the ruler 4) by means of the camera 1 and carry out a calibration of the image by automatically calculating, in a known manner, from the known distances (d1) between the graduations of the ruler 4, the resolution (Sᵢⱼ=Lᵢⱼ×Lᵢⱼ) of each pixel, i.e. the real visualised surface corresponding to a pixel of the image. In the particular example of FIG. 5a, the distance d1 between two graduations of the ruler 4 is 10 cm and covers 20 pixels, which corresponds to a resolution (Sᵢⱼ) of 5 mm×5 mm (Lᵢⱼ=5 mm).

### Step S3:

The real image of the zone to be treated (including the calibration ruler 4) is displayed on the screen 20 for the operator. This image corresponds to FIG. 5a.

The above-mentioned automatic calibration steps enable the method to be implemented regardless of the above-mentioned distance (d) between the camera 1 and the epidermis A and of the setting of the focal distance of the camera 1. When these parameters remain constant from one treatment to another, it is enough to carry out the calibration of the camera on one single occasion and it is not necessary to repeat the calibration steps prior to each treatment. It should also be underlined that the calibration of the camera 1 is optional for the implementation of the invention and is justified only for the specific embodiments in which a parameter is calculated in function of the width Lᵢⱼ or the surface Sᵢⱼ of a pixel.

Steps S4 to S10 implemented in the course of the treatment will now be described in more detail.

### Step S4:

From the above-mentioned signal 31 supplied by the source 3, the electronic treatment means 2 detect whether a shot is in progress or not. If a shot is in progress, the electronic treatment means 2 automatically carry out the following steps S5, S6, . . . .

### Step S5:

The electronic treatment means 2 trigger the acquisition of an image l(t) by means of the camera 1 and carry out a filtering of this image l(t) to detect in the image the most luminous pixel(s) forming the most luminous spot p(t) corresponding to the real irradiation spot S. In general, this most luminous spot p(t) forms a light blot, which depending on circumstances can cover several pixels of the image (this is the most frequent case corresponding to the example of the annexed drawings) or only one single pixel pᵢⱼ of the image. This light spot p(t) is not necessarily circular, but in function of the implemented filtering, the detected light blot corresponding to this light spot p(t) can have a non-circular shape.

For instance, for the filtering, a simple thresholding of the level of luminosity (level of grey in the context of a monochrome image) of the all-or-nothing type is implemented, by preserving only those pixels with a level of luminosity superior to a predefined threshold. The dimension of the light spot p(t) will thus depend on the level chosen for the filtering threshold. The higher the filtering threshold, the weaker the dimension (in number of pixels) of the light spot p(t).

The filtering threshold can be fixed and predefined. The values of the parameters can also be manually adjusted by the operator in order in particular to keep track of the depth of treatment. In another embodiment, this filtering threshold can be auto-adaptable and automatically calculated from the luminosity levels of the pixels of the image.

### Step S6:

The electronic treatment means 2 automatically calculate the energy eᵢⱼ(t) of the electromagnetic radiation for each pixel pᵢⱼ of the light spot p(t), which has been detected in the previous step by means of the following formula:

$${e}_{ij} ( t ) = \frac{P ( t ) \times τ}{n} \qquad ( 1 )$$

in which:
P(t) is the average power of the laser shot; this information is supplied to the electronic treatment means 2 by the laser source 3 (FIG. 4/signal 32);

τ is the time interval separating two successive image acquisitions; this parameter is characteristic of the camera 1 that is used and depends on the acquisition speed of the camera 1 (for instance, if the acquisition speed of the camera is 25 images per second, τ equals 40 ms).
n is the number of pixels covered by the light spot p(t).

In this embodiment, the energy eᵢⱼ(t) calculated for each pixel pᵢⱼ covered by the light spot p(t) is identical.

In a further embodiment, a calculation of the energy eᵢⱼ(t) for each pixel pᵢⱼ covered by the light spot p(t), which is weighted by the light intensity of this pixel pᵢⱼ, can also be carried out.

### Step S7:

The electronic treatment means 2 calculate for each pixel pᵢⱼ of the detected light spot p(t) the new linear energy value Eᵢⱼ(t) (energy per unit of length) by means of the following formula:

$${E}_{ij} ( t ) = {E}_{ij} ( t - 1 ) + \frac{{e}_{ij} ( t )}{{L}_{ij}} \qquad ( 2 )$$

in which:
Eᵢⱼ(t−1) is the linear energy value of the pixel pᵢⱼ prior to step S6, while specifying that during the first iteration, Eᵢⱼ(t=0) is zero;
Eᵢⱼ(t) is the value calculated during the previous step S6;
Lᵢⱼ is the width of a pixel calculated during calibration step S2.

During step S7, the electronic treatment means 2 update in the image displayed on the screen 20 the light intensity Iᵢⱼ of each pixel pᵢⱼ corresponding to the detected light spot p(t), from the new value Eᵢⱼ(t) previously calculated, this light intensity Iᵢⱼ being proportional to Eᵢⱼ(t). For instance, in the case of a monochrome image, this light intensity Iᵢⱼ is coded in grey level values from the linear energy value Eᵢⱼ that has been calculated.

For the operator Op a mapping of the electromagnetic energy for each detected irradiation spot (S) is carried out by visualising on the real image of the treated zone acquired by the camera 1 the position of each detected light spot p(t) corresponding to an irradiation spot (S), and the supplied linear energy Eᵢⱼ(t).

### Step S8:

The electronic treatment means 2 verify that the linear energy Eᵢⱼ(t) is acceptable, by comparing this value with a predefined threshold (E_{L max}), which if necessary can be regulated by the operator Op.

If the energy supplied by unit of surface Eᵢⱼ(t) is superior to this threshold, the electronic treatment means 2 control the stopping of the source 3 by means of a signal 21 (FIG. 4) and possibly trigger a warning signal 22 for the operator. This threshold is determined on a case-by-case threshold in order to stop automatically the source 3, before irreversible heat damage is caused to the dermis and the epidermis. In this case, the treatment is interrupted and the electronic treatment means 2 display for the operator Op the last updated image (step S11).

If the linear energy Eᵢⱼ(t) is inferior to this threshold, the electronic treatment means 2 carry out step S9.

In a further embodiment, several alarm thresholds can be foreseen. In this case, the above-mentioned threshold (E_{L max}) corresponds to the highest threshold. If a weaker intermediary threshold is detected as having been exceeded (without exceeding the highest threshold E_{L max}), the electronic treatment means 2 do not stop the source 3, but trigger a warning signal 22 (visual and/or audio) for the operator Op, so that the latter may react in real time, for instance by increasing the withdrawal speed of the optical fibre F to decrease the supplied linear energy dose.

### Step S9

This test is identical to the test of the above-mentioned step S4.

If a laser shot is in progress, the electronic treatment means 2 go back to step S5 (acquisition of new image l(t+1), . . . ).

If no shot is carried out by the operator Op (end of treatment sequence), the electronic treatment means 2 display on the screen 20 the last acquired image l(t) of the treated zone with the mapping of the linear energy doses Eᵤ.

By way of example, FIG. 5b shows an example of mapping of the energy doses supplied at the beginning of the treatment and FIG. 5c shows an example of mapping of the energy doses supplied at the end of the treatment.

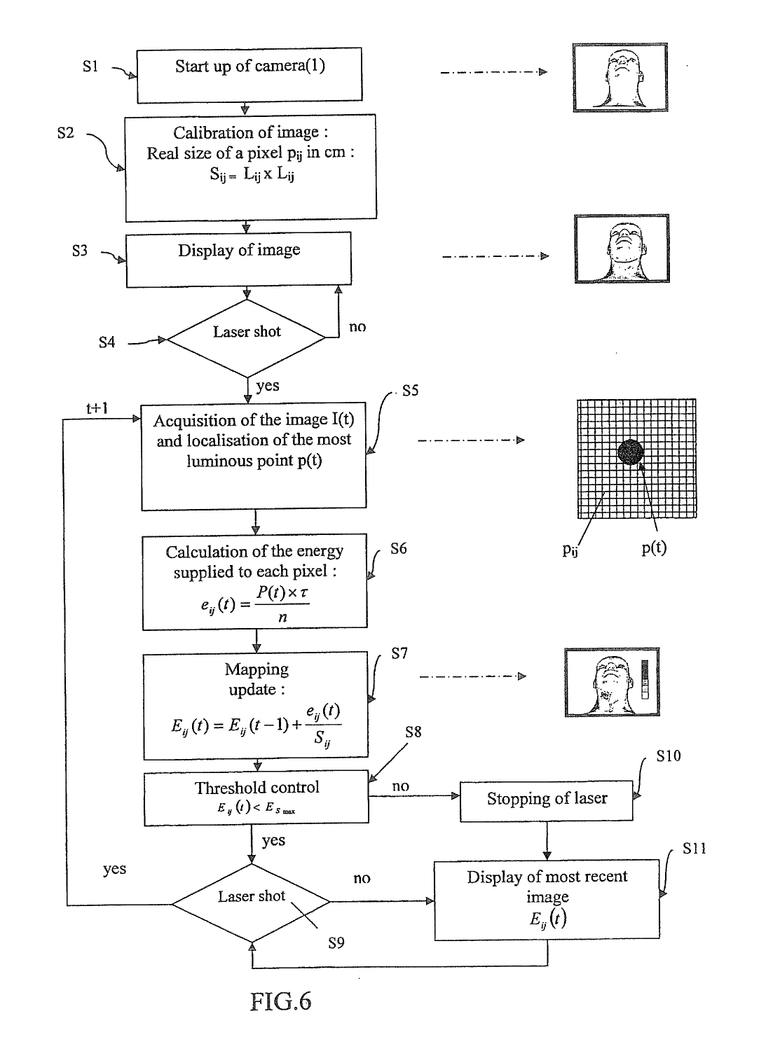

### Example of a Mapping Algorithm for a Lipolysis-Type Treatment or for a Skin Remodelling or Skin Healing Type Treatment—FIG. 6

This algorithm enables the mapping of the surface energy doses supplied during the course of a lipolysis-type treatment, during which the distal extremity of the optical fibre F is inserted into the hypodermis and is displaced in the usual manner to a layer by the operator (Op) so as to cover a surface to be treated. This algorithm also applies to any treatment by electromagnetic irradiation during which the distal extremity of the optical fibre is inserted and displaced in the sub-dermal layer SD (for instance treatment for skin remodelling or skin healing described hereinafter) or during which the distal extremity of the optical fibre is inserted and displaced in the dermis.

This algorithm is different from the above-mentioned algorithm of FIG. 5 for endovenous therapy only with regard to the calculation of step S7, which takes into account the surface Sᵢⱼ of a pixel (and not the width Lᵢⱼ of a pixel), the calculated energy value Eᵢⱼ(t) being a surface energy (energy per surface unit).

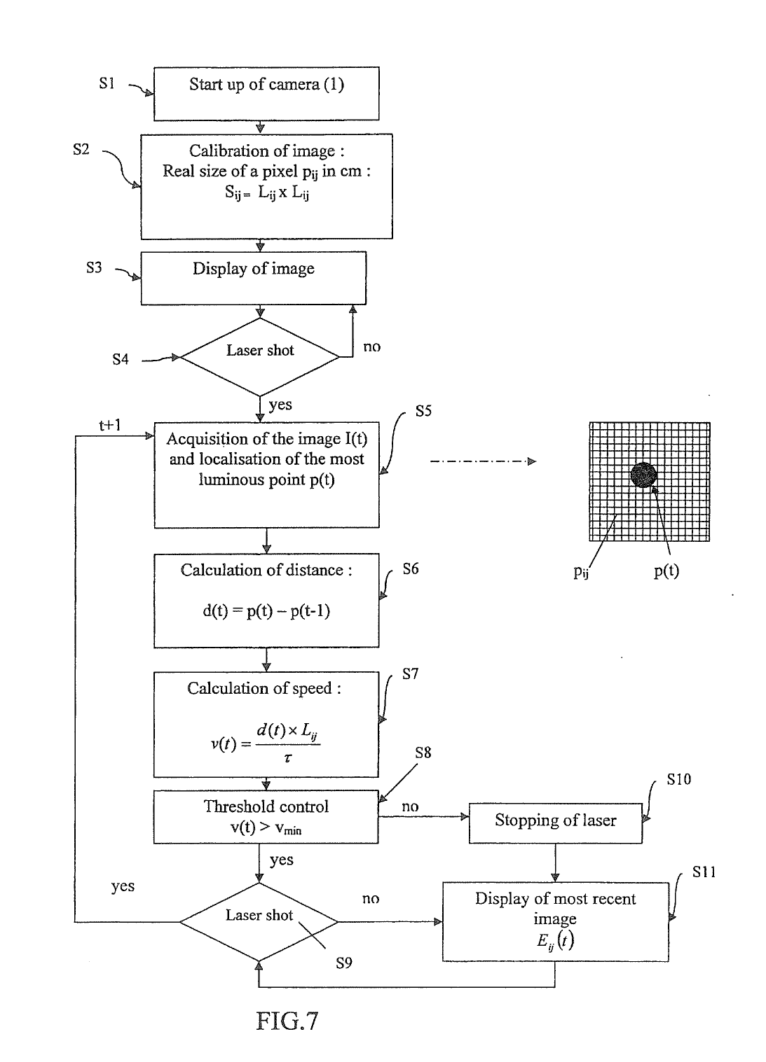

### Example of an Algorithm for the Control of the Displacement Speed of the Irradiation Spot—FIG. 7

In this algorithm, steps S1 to S3 involving the automatic calibration of the camera 1 prior to the implementation of the treatment, as well as steps. S4, S5 and S9 to S11 are identical to steps S1 to S5 or steps S9 to S11 respectively of the algorithm of FIG. 5 and will thus not be described in any more detail.

This algorithm of FIG. 7 (steps S5 to S7) enables the automatic and real-time calculation of the linear displacement speed v(t) of the irradiation spot (S) in the course of a treatment (regardless of the type of subcutaneous or intracutaneous treatment) and the automatic control (steps S8) of whether this speed v(t) is superior to a predefined minimum speed threshold (vₘᵢₙ), preferably adjustable in accordance with the treatment. If the calculated speed v(t) drops below this threshold vₘᵢₙ (corresponding to a withdrawal speed of the optical fibre by the operator that is too weak and will lead to excessive electromagnetic energy doses being supplied), the source 3 is automatically stopped by the electronic treatment means 2 (steps S10).

The calculation of the speed v(t) carried out in step S7 is obtained from two positions [lights points p(t−1) and p(t)] of the irradiation spot (S) in two successive images l(t−1) and l(t), and from the calculation of the distance d(t) (step S6) between these two positions, this distance d(t) being expressed in step S6 in the number of pixels of the image.

In a further embodiment, the treatment algorithm can be modified to be more complete and to implement for a same irradiation treatment by electromagnetic radiation both a mapping of supplied energies (eᵢⱼ(t) or Eᵢⱼ(t)) and a displacement speed v(t) control of the irradiation spot at the distal extremity of the optical fibre (merging of the algorithms of FIG. 5 or of FIG. 6 with that of FIG. 7).

The solution of the control of a treatment by subcutaneous or intracutaneous irradiation by means of electromagnetic radiation according to the invention is particularly (but not exclusively) adapted to control the new skin remodelling or skin healing method of the invention, an embodiment of which will be described hereinafter.

The main steps of this skin remodelling or skin healing method are the following.

With reference to FIG. 4, the operator Op inserts into the sub-dermal layer SD the distal extremity of the optical fibre F and displaces, with a continuous or discontinuous movement, the distal extremity of the optical fibre F in this sub-dermal layer SD by pulling on the optical fibre F. During this displacement of the optical fibre, the operator Op controls the laser source 3 to supply (in a continuous or discontinuous manner) electromagnetic energy doses into this sub-dermal layer SD at different positions of the distal extremity of the optical fibre.

The power P(t) of the source 3 or the linear displacement speed v(t) of the irradiation spot S in the sub-dermal layer SD are controlled in such a manner that the temperature in the sub-dermal layer SD is comprised between approximately 45° C. and 55° C., and preferably even between 48° C. and 52° C. A temperature superior or equal to 45° is sufficient to obtain an effective remodelling of the skin (diminution of the depth of wrinkles, remodelling of the epidermal zones that have an “orange peel skin” appearance) or an improved healing of the skin, by a heating of the collagen in the dermis and/or by a heating of the fibroblasts enabling the stimulation of collagen production in the dermis. A temperature inferior to 55° C. means that irreversible heat damage is avoided in the dermis B or in the epidermis A.

Preferably, the power P(t) of the source 3 is weak and inferior or equal to 5 W and the displacement speed v(t) is comprised between 20 mm/s and 50 mm/s.

The choice of the displacement speed v(t) for a given power P(t) depends on the wavelength of the irradiation spot (S).

By way of example, the table I below provides optimal coupling values (P(t)/V(t)) for different wavelengths.

TABLE I

| | Wavelength (nm) | | | | | |
|---|---|---|---|---|---|---|
| | 600 | 800 | 980 | 1100 | 1200 | 1400 |
| Power P(t) (W) | 5 | 5 | 5 | 5 | 5 | 5 |
| Speed v(t) (mm/s) | 50 | 20 | 10 | 15 | 20 | 60 |
| K (constant) = P(t)/v(t) | 0.1 | 0.25 | 0.5 | 0.33 | 0.25 | 0.08 |

In the range of wavelengths between 800 nm and 1200 nm, the minimum speed v(t) that should be applied is sufficiently slow to be manually implemented without difficulty by an operator Op. Outside this range, however, the minimum speed that should be applied is relatively significant, which makes the treatment riskier, since the slightest slowing down can result in a very rapid overheating of tissues. More generally, the 800 nm-1320 nm range of wavelengths is preferable for the implementation of the method. The method can nevertheless be implemented with wavelengths situated outside this range. More particularly, in a particular embodiment, the method is implemented such that the power P(t) of the electromagnetic radiation source and the linear displacement speed v(t) of the irradiation spot S in the sub-dermal layer SD respect the provision: P(t)=k.v(t), k being a predefined constant.

More particularly, k will be chosen to lie between 0.1 and 0.5, for a power expressed in Watts and a speed expressed in mm/s.

1. A control method of a treatment during which subcutaneous or intracutaneous irradiation by means of electromagnetic treatment radiation and possibly targeted electromagnetic radiation is carried out, said method comprising the following steps:
acquisition of several successive images l(t) of the treated zone by means of an external sensor, which is sensitive to the wavelength or the range of wavelengths of the electromagnetic treatment radiation or of the targeted electromagnetic radiation, the time interval (τ) between two successive images [l(t−1); l(t)] being known,
detection and localisation in each image l(t) of a light spot p(t) corresponding to the irradiation spot (S) of the electromagnetic treatment radiation or the targeted electromagnetic radiation,
calculation for each light spot p(t) of at least one of the following parameters: the energy [eᵢⱼ(t) or Eᵢⱼ(t)] supplied from the power P(t) of the electromagnetic treatment radiation and the time interval (τ) between two successive images [l(t−1); l(t)]; the displacement speed v(t) of the irradiation spot (S) from the positions of two light spots [p(t−1); p(t)] in two different images [l(t−1); l(t)] and the time interval between these two images [l(t−1); l(t)].

2. The method of claim 1, wherein the electromagnetic radiation detected by the sensor is the electromagnetic treatment radiation.

3. The method of claim 1, wherein the sensor is a CCD camera.

4. The method of claim 1, wherein the wavelength of the electromagnetic radiation detected by the sensor is comprised between 600 nm and 1400 nm.

5. The method of claim 1, wherein a mapping of the energy doses supplied during the course of the treatment is realised by associating to each detected light spot p(t) the supplied energy [eᵢⱼ(t) or Eᵢⱼ(t)] calculated for said light spot p(t).

6. The method of claim 5, wherein the mapping of the energy doses supplied throughout the treatment is displayed on a screen.

7. The method of claim 6, wherein the mapping of the supplied energy doses is displayed by being superimposed on a real image of the treated zone obtained by means of the sensor.

8. The method of claim 1, wherein, for each light spot p(t), the linear or surface energy Eᵢⱼ(t) is calculated by taking into account the real width (Lᵢⱼ) or real surface (Sᵢⱼ) of a pixel of an image l(t).

9. The method of claim 1, wherein each value calculated for the energy parameter [eᵢⱼ(t) or Eᵢⱼ(t)] is compared to at least one predefined maximum threshold and the electromagnetic treatment radiation is automatically stopped when the calculated energy value is superior to this threshold.

10. The method of claim 1, wherein each calculated value for the speed parameter v(t) is compared to at least one predefined minimum threshold (Vₘᵢₙ) and the electromagnetic treatment radiation is automatically stopped when the calculated speed value v(t) is inferior to this threshold.

11. A control system of a treatment during which subcutaneous or intracutaneous irradiation by means of electromagnetic treatment radiation and possibly targeted electromagnetic radiation is carried out, said control system comprising a sensor, which is sensitive to the wavelength or the range of wavelengths of the electromagnetic treatment radiation or of the targeted electromagnetic radiation, and which enables the acquisition of several successive images l(t) of the treated zone, with a known time interval (t) between two successive images [l(t−1); l(t)], and treatment means, which are designed automatically to process the images l(t) obtained by the sensor, so as to detect and localise in each image l(t) the irradiation spot (S) of the electromagnetic treatment radiation or the targeted electromagnetic radiation in the form of a light spot p(t) and so as to calculate for each light spot p(t) at least one of the following parameters: the energy [eᵢⱼ(t) or Eᵢⱼ(t)] supplied from the power P(t) of the electromagnetic treatment radiation and the time interval (t) between two successive images [l(t−1); l(t)]; the displacement speed v(t) of the irradiation spot (S) from the positions of two light spots [p(t−1); p(t)] in two different images [l(t−1); l(t)] and the time interval between these two images [l(t−1); l(t)].

12. The system of claim 11, wherein the sensor is a CCD camera.

13. The system of claim 11, wherein the treatment means are designed to calculate a mapping of the energy doses supplied during the course of the treatment from each detected light spot p(t) and the supplied energy [eᵢⱼ(t) or Eᵢⱼ(t)] calculated for said light spot p(t).

14. The system according of claim 13, wherein the treatment means include a screen and are designed to display on this screen the mapping of the energy doses throughout the treatment.

15. The system of claim 14, wherein the treatment means are designed to display on said screen the mapping of the energy doses by superimposition on a real image of the treated zone obtained by means of the sensor.

16. The system of claim 11, wherein the treatment means are designed to calculate, for each light spot p(t), the linear or surface energy Eᵢⱼ(t), by taking into account the real width (Lᵢⱼ) or real surface (Sᵢⱼ) of a pixel of an image l(t).

17. The system of claim 11, wherein the treatment means are designed to compare each calculated value for the energy parameter [eᵢⱼ(t) or Eᵢⱼ(t)] with at least one predefined maximum threshold and to control the automatic stopping of electromagnetic treatment radiation when the calculated energy value is superior to this threshold.

18. The system of claim 11, wherein the treatment means are designed to compare each calculated value for the speed parameter v(t) with at least one predefined minimum threshold (vₘᵢₙ) and to control the automatic stopping of electromagnetic treatment radiation when the calculated speed value is inferior to this threshold.

19. A medical device enabling a treatment by subcutaneous or intracutaneous irradiation by means of electromagnetic treatment radiation, and including at least one first source enabling the supply of an electromagnetic treatment radiation, and possibly a second source enabling the supply of a targeted electromagnetic radiation, said medical device comprising a control system as described in claim 11.

20. The medical device of claim 19, wherein the first electromagnetic radiation source communicates with the treatment means of the control system to supply to the treatment means the value of the power P(t) of the source.

21. The medical device of claim 19, wherein the treatment means of the control system are suitable for controlling the stopping of the first source of electromagnetic radiation.

22. Skin remodelling or skin healing method through irradiation by means of electromagnetic radiation, wherein the irradiation is performed in the sub-dermal layer.

23. The method of claim 22, wherein the distal extremity of an optical fibre linked to a source of electromagnetic radiation is inserted into the sub-dermal layer, wherein the distal extremity of the optical fibre is displaced in this sub-dermal layer by pulling on the optical fibre and wherein, by means of the said source of electromagnetic radiation, electromagnetic energy doses are supplied to this sub-dermal layer at different positions of the distal extremity of the optical fibre.

24. The method of claim 22, wherein the power P(t) of the source of electromagnetic radiation and the linear displacement speed v(t) of the irradiation spot (S) in the sub-dermal layer respect the provision: P(t)=k.v(t), k being a predefined constant.

25. The method of claim 24, wherein k lies between 0.1 and 0.5 for a power P(t) expressed in Watt and a speed v(t) expressed in mm/s.

26. The method of claim 22, wherein the wavelength of the electromagnetic radiation lies between 800 nm and 1320 nm.

27. The method of claim 22, wherein the power of the source of electromagnetic radiation is inferior or equal to 5 W.

28. The method of claim 22, wherein the linear displacement speed v(t) of the distal extremity of the optical fibre is inferior or equal to 50 mm/s, and preferably lies between 20 mm/s and 50 mm/s.

29. A use of the control method described in claim 1 to control a lipolysis treatment or endovenous therapy.

30. A use of the control method described in claim 1 to control an epidermal remodelling or skin healing through irradiation by means of electromagnetic radiation, wherein the irradiation is performed in the sub-dermal layer.

31. A use of the control system described in claim 11 to control a lipolysis treatment or endovenous therapy.

32. A use of the control system described in claim 11 to control an epidermal remodelling or skin healing through irradiation by means of electromagnetic radiation, wherein the irradiation is performed in the sub-dermal layer.
